(19) United States
(12) Patent Application Publication
(10) Pub. No.: US 2006/0233731 A1
(43) Pub. Date: Oct. 19, 2006

(54) Use, for the cosmetic treatment of keratin materials, of a film-forming composition comprising an electrophilic monomer and a non-silicone polymer, conferring a soft coating

(76) Inventors: Gabin Vic, Venette (FR); Gaelle Brun, Paris (FR); Aude Livoreil, Paris (FR); Luc Gourlaouen, Asnieres (FR); Franck Giroud, Clichy (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/247,583

(22) Filed: Oct. 12, 2005

Related U.S. Application Data
(60) Provisional application No. 60/646,491, filed on Jan. 25, 2005.

(30) Foreign Application Priority Data
Oct. 13, 2004 (FR) 04 10808

(51) Int. Cl. A61K 8/81; A61K 8/92

(52) U.S. Cl. 424/70.7

(57) ABSTRACT

The present invention relates to the use, for the cosmetic treatment of keratin materials, of a film-forming composition comprising, in a cosmetically acceptable medium, at least one electrophilic monomer and at least one non-silicone polymer such that the film obtained from the composition by drying at ambient temperature and with a relative humidity of approximately 50% has a Young's modulus of between 1 and 100 MPa, measured for a thickness of 0.5 mm and with a tensile speed of 20 mm/min.

The present invention relates to the use, for the cosmetic treatment of keratin materials, of a film-forming composition comprising at least one electrophilic monomer and at least one specific non-silicone polymer, and to a method of cosmetic treatment using such a composition.

In the cosmetics field, attempts are being made to modify the superficial properties of keratin materials, and more particularly of keratin fibres such as the hair, for example so as to give the hair a conditioning effect such as softness or sheen. To do this, use is generally made of cosmetic compositions based on conditioning agents such as silicones or polymers that have a high affinity for keratin materials, and in particular for the hair.

However, the coating of the keratin fibres such as the hair, obtained with these compositions, often has an unpleasant tacky feel and also the drawback of transfer, for example, when an individual runs a hand through the hair. This transfer phenomenon leaves a feeling of dirty or tacky hair.

In addition, these conditioning agents have a tendency to disappear in the course of washing with shampoos, making it necessary to renew the applications of the compositions to the hair.

In order to increase the persistence of deposit of polymers, it is possible to envisage performing a free-radical polymerization of certain monomers directly on the hair. However, a considerable degradation of the hair fibres is noted, probably associated with the polymerization initiators, and the hair thus treated is difficult to disentangle.

The applicant has found, surprisingly, that by using the combination of at least one electrophilic monomer as described below with at least one specific non-silicone polymer, it is possible to overcome the draw-backs of the coatings of the prior art and to obtain improved conditioning and an improved sheen of the hair, in a long-lasting manner.

The specific non-silicone polymer is selected in such a way that combination thereof with at least one electrophilic monomer in a composition results in the formation of a film, after drying of the composition at ambient temperature and a relative humidity of approximately 50%, which has a Young's modulus between 1 and 100 MPa, measured for a thickness of 0.5 mm and with a tensile speed of 20 mm/min. This Young's modulus value results in a “soft” coating of the keratin materials being obtained.

The composition comprising such a combination makes it possible to maintain the softness and the sheen given to the hair by said composition, without re-application, even after several washes of the hair.

The application of a composition comprising such a combination results in the formation, in situ, of a non-tacky and shiny coating that is persistent, in particular with respect to washing.

In addition, when it is applied to the hair, said hairs remain, surprisingly, completely individualized and can be styled without any problem.

A subject of the invention is therefore the use, for the cosmetic treatment of keratin materials, of a film-forming composition comprising, in a cosmetically acceptable medium, at least one electrophilic monomer and at least one non-silicone polymer as defined below.

Another subject of the present invention consists of a method of cosmetic treatment of keratin materials using said composition.

A subject of the invention is also a kit comprising a first composition containing at least one electrophilic monomer and, optionally, at least one anionic and/or free-radical polymerization inhibitor, and also a second composition comprising, in a cosmetically acceptable medium, at least one non-silicone block copolymer as defined below.

Other subjects, characteristics, aspects and advantages of the invention will emerge even more clearly on reading the description and the example that follows.

According to the invention, the film-forming composition comprises, in a cosmetically acceptable medium, at least one electrophilic monomer and at least one non-silicone polymer such that the film obtained from the composition by drying at ambient temperature and with a relative humidity of approximately 50% has a Young's modulus of between 1 and 100 MPa, measured for a thickness of 0.5 mm and with a tensile speed of 20 mm/min.

For the purpose of the present invention, the term “ambient temperature” is intended to mean a temperature included in the range of from 20 to 24° C.

The non-silicone polymer has a main chain comprising carbon and hydrogen atoms, which chain can be interrupted with one or more hetero atoms such as O, N, P and S, and which can comprise one or more end-of-chain or lateral functions.

The non-silicone polymer used in the present invention may be dendritic, linear, branched, a block polymer, a polymer in the form of a comb or a polymer in the form of a star. It may exhibit one or more types of repeat units, and it may be a random, block or alternating copolymer or homopolymer. It is preferably a block copolymer.

The polymer comprises at least five repeat units linked via covalent bonds.

For the purpose of the present invention, the term “non-silicone” is intended to mean a polymer that contains no —Si—O—Si— chain.

The homopolymers or copolymers that may be used in the present invention can solubilize or disperse spontaneously or via neutralization in water. They may consist of at least two blocks that are hydrophilic in nature and have a different chemical composition, or else of at least one block that is hydrophilic in nature and at least one block that is hydrophobic in nature. They may be anionic, cationic, nonionic or amphoteric.

When they solubilize or disperse spontaneously in an anhydrous medium, the homopolymers or copolymers that may be used in the present invention may consist of at least two blocks that are hydrophobic in nature and have a different chemical composition, or else of at least one block that is hydrophobic in nature and at least one block that is hydrophilic in nature. They may be anionic, cationic, nonionic or amphoteric.

The expression “water-soluble or water-dispersible or liposoluble homopolymer or copolymer” is intended to mean a homopolymer or copolymer which, at the concentration of 0.1% by weight in terms of active material in water or in an anhydrous solvent, at 25° C., gives, spontaneously or after neutralization with an acid or a base, a macroscopically homogeneous solution or suspension that is transparent or translucent, i.e. that exhibits a transmittance at a wavelength of 500 nm, through a sample 1 cm thick.

The expression “block that is hydrophilic in nature” is intended to mean a block consisting of at least 75 mol % of monomers that are water-soluble and/or can be made water-soluble by neutralization. The block that is hydrophilic in nature comprises at most 25 mol % of non-water-soluble monomers, preferably at most 10 mol %, and even better still at most 5 mol %.

The expression “block that is hydrophobic in nature” is intended to mean a block consisting of at least 75 mol % of non-water-soluble monomers. The block that is hydrophobic in nature comprises at most 25 mol % of water-soluble monomers, preferably at most 10 mol %, and even better still at most 5 mol %.

By way of examples of monomers that can be used in the polymers of the invention, mention may in particular be made of those disclosed in application FR 2 840 205.

Preferably, the non-silicone polymers used in the invention are amphiphilic linear block copolymers.

The water-soluble monomers that form the hydrophilic block(s) of the block copolymers that can be used in the present invention may be anionic, nonionic or cationic in nature and may be used alone or in the form of a mixture containing two or more different monomers.

By way of examples of anionic water-soluble monomers, mention may be made of carboxylic acids with an ethylenic unsaturation, such as acrylic acid, methacrylic acid, itaconic acid, fumaric acid, crotonic acid and maleic acid, 2-acrylamido-2-methylpropane-sulphonic acid, styrenesulphonic acid, vinylsulphonic acid and vinylphosphonic acid.

The nonionic water-soluble monomers encompass, among others, acrylamide, C₁-C₆ N-alkylated or C₁-C₃ N,N-dialkylated acrylamides, polyethylene glycol acrylate, polyethylene glycol methacrylate, N-vinylacetamide, N-methyl-N-vinylacetamide, N-vinylformamide, N-methyl-N-vinylformamide, N-vinyl lactams containing a cyclic group having from 4 to 9 carbon atoms, vinyl alcohol (copolymerized in the form of vinyl acetate and then hydrolysed), ethylene oxide, hydroxyethyl acrylate, hydroxypropyl acrylate, hydroxyethyl methacrylate and hydroxypropyl methacrylate.

Finally, the cationic water-soluble monomers encompass, for example, dimethyldiallylammonium chloride, methyl-vinylimidazolium chloride, 2-vinylpyridine, 4-vinyl-pyridine, 2-methyl-5-vinylpyridine, N—(C₁-C₄ alkyl)-4-vinylpyridinium halides such as N-methyl-4-vinyl-pyridinium iodide, al vinylamine, and the monomers of formula
H₂C═CX₁—CO—X₂
in which

X₁ represents a hydrogen atom or methyl group,

X₂ represents a linear or branched C₁-C₆ hydrocarbon-based group having at least one primary, secondary or tertiary amine function or at least one quaternary nitrogen atom, or a group of formula NHR₂ or of formula NX₃X₄ where X₃ and X₄ each represent, independently of one another, a linear or branched C₁-C₆ hydrocarbon-based group carrying at least one primary, secondary or tertiary amine function or at least one quaternary nitrogen atom.

The water-insoluble (non-water-soluble) monomers that form the hydrophobic block(s) of the block copolymers are preferably chosen from vinylaromatic monomers, such as styrene and its alkylated derivatives, for instance 4-butylstyrene, α-methylstyrene and vinyltoluene, dienes such as butadiene and 1,3-hexadiene, and alkylated derivatives of dienes, such as isoprene and dimethylbutadiene, chloroprene, C₁-C₁₀ alkyl, C₆-C₁₀ aryl or C₆-C₁₀ aralkyl acrylates and C₁-C₁₀ alkyl, C₆-C₁₀ aryl or C₆-C₁₀ aralkyl methacrylates, for instance methyl(meth)acrylate, ethyl(meth)acrylate, n-butyl(meth)acrylate, 2-ethylhexyl(meth)acrylate, tert-butyl(meth)acrylate, isobornyl(meth)acrylate, phenyl(meth)acrylate or benzyl(meth)acrylate, vinyl acetate, vinyl ethers of formula CH₂═CH—O—R″ and allyl ethers of formula CH₂═CH—CH₂—O—R″, where R″ represents a C₁-C₆ alkyl group, acrylonitrile, vinyl chloride, vinylidene chloride, caprolactone, ethylene, propylene, fluorinated vinyl monomers or vinyl monomers comprising a perfluorinated chain, such as fluoroalkyl acrylates and methacrylates or alkyl α-fluoroalkylates.

The non-silicone polymer(s) as described above is or are present in the cosmetic compositions of the invention at a concentration of between 0.05% and 99% by weight, preferably of between 0.1% and 95% by weight, and even better still of between 0.2 and 30% by weight, relative to the total weight of the composition.

The term “electrophilic monomer” is intended to mean a monomer capable of polymerizing by anionic polymerization in the presence of a nucleophilic agent such as, for example, the hydroxyl ions (OH⁻) contained in water.

The term “anionic polymerization” is intended to mean the mechanism defined in the work “Advanced Organic Chemistry”, Third Edition by Jerry March, pages 151 to 161.

The electrophilic monomer(s) present in the composition of the invention may be chosen from:

(i) benzylidene malononitrile derivatives (A), 2-(4-chlorobenzylidene)malononitrile (A1), ethyl 2-cyano-3-phenylacrylate (B), ethyl 2-cyano-3-(4-chlorophenyl)acrylate (B1), as described in Sayyah, J. Polymer Research, 2000, p 97:

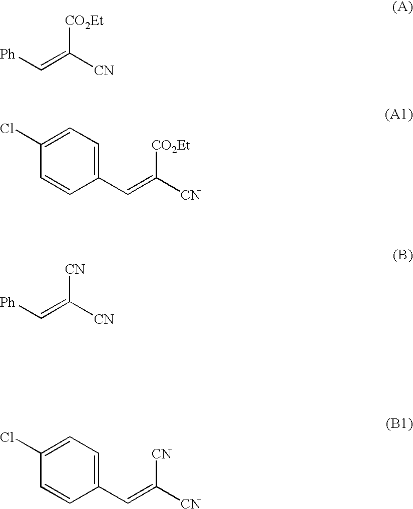

(ii) methylidenemalonate derivatives such as:

diethyl 2-methylenemalonate (C) as described by Hopff, Makromoleculare Chemie, 1961, p 95, by De Keyser, J. Pharm. Sci, 1991, p 67 and by Klemarczyk, Polymer, 1998, p 173:

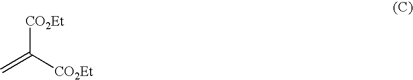

ethyl 2-ethoxycarbonylmethyleneoxycarbonyl acrylate (D) as described by Breton, Biomaterials, 1998, p 271 and Couvreur, Pharmaceutical Research, 1994, p 1270:

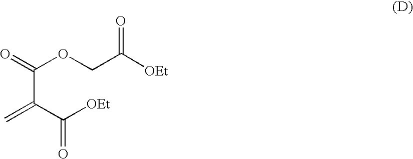

(iii) itaconate and itaconimide derivatives such as:

dimethyl itaconate (E) as described by Bachrach, European Polymer Journal, 1976, p 563:

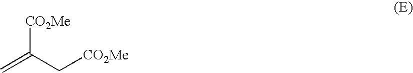

N-butylitaconimide (F), N-(4-tolyl)itaconimide (G), N-(2-ethylphenyl)itaconimide (H), N-(2,6-diethylphenyl)-itaconimide (I), as described by Wanatabe, J. Polymer Science: Part A: Polymer Chemistry, 1994, p 2073: 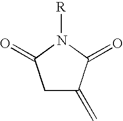
R=Bu(F), 4-tolyl (G), 2-ethylphenyl (H), 2,6-diethylphenyl (I)

(iv) methyl α-(methylsulphonyl)acrylate derivatives (K), ethyl α-(methylsulphonyl)acrylate derivatives (L), methyl α-(tert-butylsulphonyl)acrylate derivatives (M), tert-butyl α-(methylsulphonyl)acrylate derivatives (N), tert-butyl α-(tert-butylsulphonyl)acrylate derivatives (O) as described by Gipstein, J. Org. Chem., 1980, p 1486, and the derivatives 1,1-bis(methylsulphonyl)-ethylene (P), 1-acetyl-1-methylsulphonylethylene (Q), methyl α-(methylsulphonyl) vinyl sulphonate (R), α-methylsulphonylacrylonitrile (S), as described by Shearer, U.S. Pat. No. 2,748,050:

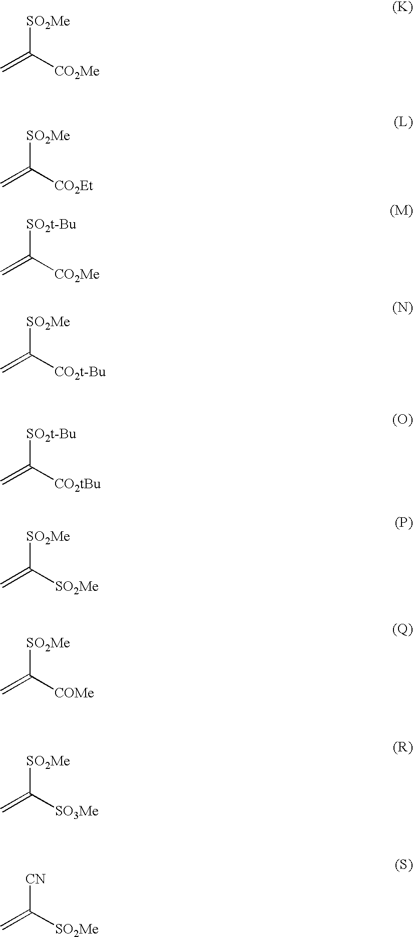

(v) the methyl vinyl sulphone (T) and phenyl vinyl sulphone (U) derivatives as described by Boor, J. Polymer Science, 1971 p 249:

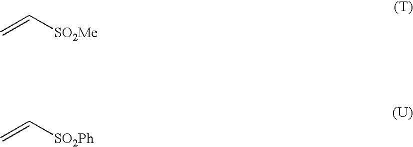

(vi) the phenyl vinyl sulphoxide (V) derivative as described by Kanga, Polymer Preprints (ACS, Division of Polymer Chemistry), 1987, p 322:

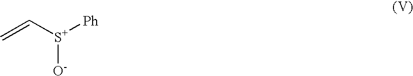

(vii) the 3-methyl-N-(phenylsulphonyl)-1-aza-1,3-butadiene derivative (W) as described by Bonner, Polymer Bulletin, 1992, p 517:

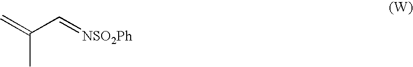

(viii) acrylate and acrylamide derivatives such as:

N-propyl-N-(3-triisopropoxysilylpropyl)acrylamide (X) and N-propyl-N-(3-triethoxysilylpropyl)acrylamide (Y) as described by Kobayashi, Journal of Polymer Science, Part A: Polymer Chemistry, 2005, p 2754:

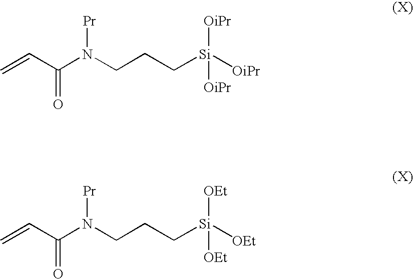

2-hydroxyethyl acrylate (Z) and 2-hydroxyethyl methacrylate (AA), as described by Rozenberg, International Journal of Plastics Technology, 2003, p 17:

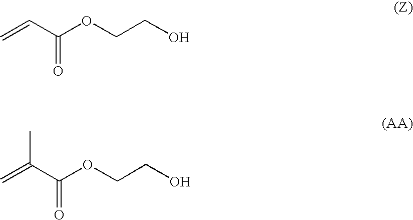

N-butyl acrylate (AB) as described by Schmitt, Macromolecules, 2001, p 2115, and tert-butyl acrylate (AC) as described by Ishizone, Macromolecules, 1999, p 955:

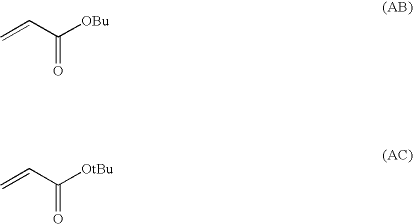

The electrophilic, or electron-withdrawing, monomer that is useful in the present invention may be cyclic or linear. When it is cyclic, the electron-withdrawing group is preferably exocyclic, i.e. it is not an integral part of the cyclic structure of the monomer.

According to a particular embodiment, these monomers have at least two electron-withdrawing groups.

By way of example of electrophilic monomers having at least two electron-withdrawing groups, mention may be made of the monomers of formula (I):

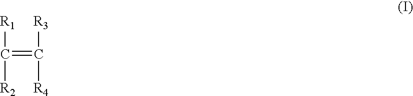

in which:

R₁ and R₂ each denote, independently of one another, a group with little or no electron-withdrawing effect (with little or no inductive withdrawing effect) such as:
a hydrogen atom,
a saturated or unsaturated, linear, branched or cyclic hydrocarbon-based group preferably containing from 1 to 20, even better still from 1 to 10, carbon atoms, and optionally containing one or more nitrogen, oxygen or sulphur atoms, and optionally substituted with one or more groups chosen from —OR, —COOR, —COR, —SH, —SR, —OH, and halogen atoms,
a modified or unmodified polyorganosiloxane residue,
a polyoxyalkylene group,

R₃ and R₄ each denote, independently of one another, an electron-withdrawing group (or group with inductive withdrawing effect) preferably chosen from the groups —N(R)₃⁺, —S(R)₂⁺, —SH₂⁺, —NH₃⁺, —NO₂, —SO₂R, ——C≡N, —COOH, —COOR, —COSR, —CONH₂, —CONHR, —F, —Cl, —Br, —I, —OR, —COR, —SH, —SR and —OH, linear or branched alkenyl groups, linear or branched alkynyl groups, mono- or polyfluoro(C₁-C₄)alkyl groups, aryl groups such as phenyl, or aryloxy groups such as phenyloxy,

R denotes a saturated or unsaturated, linear, branched or cyclic hydrocarbon-based group preferably containing from 1 to 20, even better still from 1 to 10, carbon atoms, and optionally containing one or more nitrogen, oxygen or sulphur atoms, and optionally substituted with one or more groups chosen from —OR′, —COOR′, —COR′, —SH, —SR′, —OH, halogen atoms, and a residue of a polymer that can be obtained by free-radical polymerization, by polycondensation or by ring opening, R′ denoting a C₁-C₁₀ alkyl group.

The expression “electron-withdrawing group or group with inductive withdrawing effect (—I)” is intended to mean any group that is more electronegative than carbon. Reference may be made to the work PR Wells Prog. Phys. Org. Chem., Vol 6, 111 (1968).

The expression “group with little or no electron-withdrawing effect” is intended to mean any group whose electronegativity is less than or equal to that of carbon.

The alkenyl or alkynyl groups preferably have 2 to 20 carbon atoms, even better still from 2 to 10 carbon atoms.

As a saturated or unsaturated, linear, branched or cyclic hydrocarbon-based group preferably containing from 1 to 20 carbon atoms, mention may in particular be made of linear or branched alkyl, alkenyl or alkynyl groups, such as methyl, ethyl, n-butyl, tert-butyl, isobutyl, pentyl, hexyl, octyl, butenyl or butynyl; cycloalkyl groups or aromatic groups.

As a substituted hydrocarbon-based group, mention may, for example, be made of hydroxyalkyl or polyhaloalkyl groups.

By way of examples of unmodified polyorganosiloxane, mention may in particular be made of polyalkylsiloxanes such as polydimethylsiloxanes, polyarylsiloxanes such as polyphenylsiloxanes, and polyarylalkylsiloxanes such as polymethylphenylsiloxanes.

Among modified polyorganosiloxanes, mention may in particular be made of polydimethylsiloxanes comprising polyoxyalkylene and/or siloxy and/or silanol and/or amine and/or imine and/or fluoroalkyl groups.

Among polyoxyalkylene groups, mention may in particular be made of polyoxyethylene groups and polyoxypropylene groups having preferably 1 to 200 oxyalkylenated units. Among mono- or polyfluoroalkyl groups, mention may in particular be made of groups such as —(CH₂)n-(CF₂)m-CF₃ or —(CH₂)n-(CF₂)m-CHF₂ with n=1 to 20 and m=1 to 20.

The substituents R₁ to R₄ may optionally be substituted with a group having a cosmetic activity. The cosmetic activities particularly used are obtained from groups with colouring, antioxidant, UV-screening and conditioning functions.

By way of examples of a group with a colouring function, mention may in particular be made of azo, quinone, methine, cyanomethine and triarylmethane groups.

By way of examples of a group with an antioxidant function, mention may in particular be made of groups of butylhydroxyanisole (BHA), butylhydroxytoluene (BHT) or vitamin E type.

By way of examples of a group with a UV-screening function, mention may in particular be made of groups of benzophenone, cinnamate, benzoate, benzylidene-camphor and dibenzoylmethane types.

By way of examples of a group with a conditioning function, mention may in particular be made of cationic and fatty ester-type groups.

Among the monomers mentioned above, preference is given to monomers of the cyanoacrylate family and derivatives thereof of formula (II):

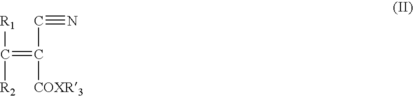

X denoting NH, S or O,

R₁ and R₂ having the same meanings as above,

it being possible for R′₃ to denote a hydrogen atom or a group R as defined for formula (I).

Preferably, X denotes O.

As compounds of formula (II), mention may be made of the monomers:

a) belonging to the family of C₁-C₂₀ polyfluoroalkyl 2-cyanoacrylates, such as:

2-cyano-2-propenoic acid 2,2,3,3-tetrafluoropropyl ester of formula:

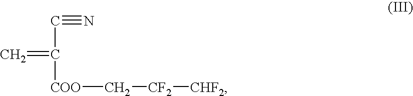

or else 2-cyano-2-propenoic acid 2,2,2-trifluoroethyl ester of formula:

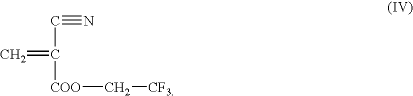

b) (C₁-C₁₀)alkyl or (C₁- C4) alkoxy(C₁-C₁₀)alkyl cyano-acrylates.

Mention may more particularly be made of ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyano-acrylate, n-octyl 2-cyanoacrylate and isoamyl cyano-acrylate.

In the context of the invention, use is preferably made of the monomers b).

The monomers that are more particularly preferred are those of formula (V) and mixtures thereof:

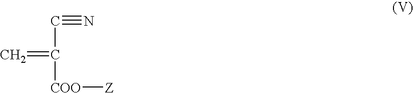

in which: Z=—(CH₂)₇—CH₃,
—CH(CH₃)—(CH₂)₅—CH₃,
—CH₂—CH(C₂H₅)—(CH₂)₃—CH₃,
—(CH₂)₅—CH(CH₃)—CH₃,
—(CH₂)₄—CH(C₂H₅)—CH₃.

The monomers used in accordance with the invention may be covalently attached to supports such as polymers, oligomers or dendrimers. The polymer or the oligomer may be linear, branched, as a comb or as a block. The distribution of the monomers of the invention on the polymeric, oligomeric or dendritic structure may be random, at the end position or in the form of blocks.

According to the present invention, the monomers are preferably chosen from monomers capable of polymerizing on keratin fibres under cosmetically acceptable conditions. In particular, the polymerization of the monomer preferably takes place at a temperature of less than or equal to 80° C., preferably between 10 and 80° C., preferably 20 to 80° C., which does not prevent the application from being finished with drying under a hood, blow-drying, or the use of a flat heating iron or curling tongs.

The compositions used in accordance with the invention generally have a concentration of electrophilic monomer according to the invention of between 0.001 and 80% by weight, and more particularly between 0.1 and 40%, and even more preferably between 1 and 20% by weight, relative to the total weight of the composition.

The term “cosmetically acceptable medium” is intended to mean a medium that is compatible with keratin materials such as the hair.

The cosmetically acceptable medium is preferably anhydrous. The term “anhydrous medium” is intended to mean a medium containing less than 1% by weight of water relative to the total weight of the composition.

The cosmetically acceptable medium is preferably chosen from organic oils; silicones such as volatile silicones, amino or non-amino silicone gums or oils and mixtures thereof; mineral oils; plant oils such as olive oil, castor oil, rapeseed oil, coconut oil, wheat germ oil, sweet almond oil, avocado oil, macadamia oil, apricot oil, safflower oil, candlenut oil, camelina oil, tamanu oil and lemon oil; waxes; or alternatively organic compounds such as C₅-C₁₀ alkanes, acetone, methyl ethyl ketone, esters of C₁-C₂₀ acids and of C₁-C₈ alcohols such as methyl acetate, butyl acetate, ethyl acetate and isopropyl myristate, dimethoxyethane, diethoxyethane, C₁₀-C₃₀ fatty alcohols such as lauryl alcohol, cetyl alcohol, stearyl alcohol and behenyl alcohol, C₁₀-C₃₀ fatty acids such as lauric acid and stearic acid, C₁₀-C₃₀ fatty amides such as lauric diethanolamide, and esters of C₁₀-C₃₀ fatty alcohols such as C₁₀-C₃₀ fatty alcohol benzoates, and mixtures thereof.

Preferably, the organic compounds are chosen from compounds that are liquid at a temperature of 25° C. and at 10⁵ Pa (760 mmHg).

Polymerization inhibitors, and more particularly anionic and/or free-radical polymerization inhibitors, may also be introduced into the compositions, in order to increase the stability of the composition over time. In a non-limiting manner, the following polymerization inhibitors may be mentioned: sulphur dioxide, nitric oxide, lactone, boron trifluoride, hydroquinone and derivatives thereof such as hydroquinone monoethyl ether, tert-butylhydroquinone (TBHQ), benzoquinone and derivatives thereof such as duroquinone, catechol and derivatives thereof such as t-butylcatechol and methoxycatechol, anisole and derivatives thereof such as methoxyanisole, hydroxyanisole or butylhydroxy-anisole, pyrogallol, 2,4-dinitrophenol, 2,4,6-tri-hydroxybenzene, p-methoxyphenol, hydroxybutyltoluene, alkyl sulphates, alkyl sulphites, alkyl sulphones, alkyl sulphoxides, alkyl sulphides, mercaptans and 3-sulpholene, and mixtures thereof. The alkyl groups preferably denote groups containing 1 to 6 carbon atoms.

It is also possible to use mineral or organic acids, the latter containing one or more carboxylic or sulphonic groups, with a pKa of between 0 and 6, such as phosphoric acid, hydrochloric acid, nitric acid, benzenesulphonic or toluenesulphonic acid, sulphuric acid, carbonic acid, hydrofluoric acid, acetic acid, formic acid, propionic acid, benzoic acid, mono-, di or trichloroacetic acid, salicylic acid and trifluoro-acetic acid.

The amount of inhibitor may range from 10 ppm to 20%, more preferably from 10 ppm to 5%, and even more preferably from 10 ppm to 1% by weight relative to the total weight of the composition.

The compositions in accordance with the invention may also contain at least one agent normally used in cosmetics, such as, for example, reducing agents, fatty substances, plasticizers, softeners, antifoams, moisturizers, pigments, clays, mineral fillers, UV-screening agents, mineral colloids, peptizers, solubilizing agents, fragrances, preserving agents, anionic, cationic, nonionic or amphoteric surfactants, fixing or non-fixing polymers, polyols, proteins, vitamins, direct dyes or oxidation dyes, pearlescent agents, propellent gases and mineral or organic thickeners such as benzylidene sorbitol and N-acylamino acids.

These agents may optionally be encapsulated. The capsule may be of polycyanoacrylate type.

For the cosmetic treatment of keratin materials, and more particularly of the hair, the composition is preferably used in the presence of a nucleophilic agent.

The method of cosmetic treatment according to the invention comprises the application of a composition as defined above to keratin materials, in the presence of a nucleophilic agent as defined below.

The nucleophilic agents capable of initiating the anionic polymerization are systems that are known in themselves, and are capable of generating a carbanion on contact with an electrophilic monomer. The term “carbanion” is intended to mean the chemical species defined in “Advanced Organic Chemistry, Third Edition”, by Jerry March, page 141.

The nucleophilic agents may consist of a molecular compound, an oligomer, a dendrimer or a polymer containing nucleophilic functions. In a non-limiting manner, nucleophilic functions that may be mentioned include the following functions: R₂N⁻, NH₂⁻, Ph₃C⁻, R₃C⁻, PHNH⁻, pyridine, ArS⁻, R—C≡C⁻, RS⁻, SH⁻, RO⁻, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄⁻ and H₂O, Ph representing a phenyl group; Ar representing an aryl group and R representing a C₁-C₁₀ alkyl group.

The nucleophilic agents that are particularly preferred are hydroxyl ions, in particular those present in water. This water can be provided by prior moistening of the keratin materials.

According to a preferred embodiment, the composition comprising the electrophilic monomer is free of nucleophilic agent.

According to another preferred embodiment, the nucleophilic agent is provided by a second composition, applied onto or under the deposit formed by the application of the composition comprising the electrophilic monomer.

It is also possible, in order to modulate the reaction kinetics, to moisten the keratin materials, and in particular the keratin fibres, beforehand using an aqueous solution whose pH has been adjusted using a base, an acid or an acid/base mixture. The acid and/or the base may be mineral or organic.

It is also possible to modulate the anionic polymerization kinetics by preimpregnating the keratin materials, and in particular the keratin fibres, using a nucleophilic agent other than water. The nucleophilic agent may be used pure, as a solution, in the form of an emulsion or may be encapsulated.

It is also possible, in order to modulate the anionic polymerization kinetics, to increase the nucleophilicity of the keratin materials, and in particular of the keratin fibres, by chemical conversion of the keratin material.

By way of example of chemical conversion, mention may be made of the reduction of the disulphide bridges of which keratin is partly composed, to thiols, before application of the composition of the invention. In a non-exhaustive manner, as reducing agents for the disulphide bridges of which keratin is partly composed, mention may be made of the following compounds:
anhydrous sodium thiosulphate,
powdered sodium metabisulphite,
thiourea,
ammonium sulphite,
thioglycolic acid,
thiolactic acid,
ammonium thiolactate,
glyceryl monothioglycolate,
ammonium thioglycolate,
thioglycerol,
2,5-dihydroxybenzoic acid,
diammonium dithioglycolate,
strontium thioglycolate,
calcium thioglycolate,
zinc formosulphoxylate,
isooctyl thioglycolate,
dl-cysteine,
monoethanolamine thioglycolate.

In order to modulate the anionic polymerization kinetics, and more particularly to reduce the rate of polymerization of the monomers of the invention, it is possible to increase the viscosity of the composition. To do this, one or more polymers that have no reactivity towards the monomers in accordance with the invention may be added to the composition of the invention. In this context, mention may be made, in a non-exhaustive manner, of poly(methyl methacrylate) (PMMA) or alternatively cyanoacrylate-based copolymers as described in U.S. Pat. No. 6,224,622.

In order to improve, inter alia, the adhesion of the poly(cyanoacrylate) formed in situ, the fibre may be pretreated with polymers of any type, or a hair treatment may be performed before application of the composition of the invention, for instance a direct dyeing or oxidation dyeing, permanent-waving or hair relaxing operation.

The application of the compositions as described above may or may not be followed by rinsing.

The compositions may be in the form of a lotion, a spray or a mousse, and may be applied in the form of a shampoo or a conditioner.

Another subject of the invention is a composition comprising at least one electrophilic monomer and at least one non-silicone block copolymer as described above.

For the purpose of the present invention, the term “block copolymer” is intended to mean any copolymer containing at least two different blocks. Preferably, the block polymers of the invention are linear. Such polymers are described above.

A subject of the invention is also a kit comprising a first composition containing at least one electrophilic monomer and, optionally, at least one anionic and/or free-radical polymerization inhibitor, and also a second composition comprising, in a cosmetically acceptable medium, at least one non-silicone block copolymer as described above.

Another subject of the present invention is a method of cosmetic treatment of keratin materials, comprising at least two steps, one step comprising the application of the non-silicone polymer as defined above, and another step comprising the application of at least one electrophilic monomer as defined above, the order of the steps being irrelevant.

In a particular embodiment of the invention, the application of the non-silicone polymer is carried out before the application of at least one electrophilic monomer.

The following example is given by way of illustration of the present invention.

In the following example, all the amounts are indicated as per cent by weight of active material relative to the total weight of the composition, unless otherwise indicated.

### EXAMPLES

### Example 1

The following composition was prepared in accordance with the invention:

| | |
|---|---|
| 2-Octyl cyanoacrylate | 60% |
| Poly(vinyl laurate) | 39% |
| Monoethanolamine | 1% |

The composition is deposited onto a Teflon matrix. The film is dried at ambient temperature and at a relative humidity of 50%. The thickness of the film obtained is 0.5 mm.

Young's modulus is subsequently measured with a Lloyd model RLSK device, for a tensile speed of 20 mm/min. The value is approximately 10 MPa.

The composition applied to the hair and dried confers a shampoo-fast soft coating.

### Example 2

This example demonstrates the soft nature of the coating.

The following composition is prepared in accordance with the invention:

| | |
|---|---|
| Methylheptyl cyanoacrylate (1) | 10% |
| DC245 Fluid from Dow Corning | 42% |
| DC 1501 Fluid from Dow Corning | 42% |
| Resin PA 18 (2) | 5% |
| Monoethanolamine | 1% |

(1) sold by the company Chemence
(2) sold by the company Chevron Phillips Chemical Company

This composition is applied to clean moist hair. 0.16 g of formula is applied per g of hair. The hair is subsequently dried for 30 minutes under a hood.

The measurement of the Young's modulus of the coating formed directly on the hair is performed by means of nanoindentation by atomic force microscopy (AFM).

The value obtained for the Young's modulus is 95 MPa, which clearly corresponds to a soft coating.

### Examples 3 to 9

Examples 3 to 9 below illustrate other compositions in accordance with the invention.

### Example 3

| | |
|---|---|
| Methylheptyl cyanoacrylate (1) | 10% |
| DC245 Fluid from Dow Corning | 25% |
| DC 1501 Fluid from Dow Corning | 25% |
| Poly(vinyl laurate) | 39% |
| Monoethanolamine | 1% |

(1) sold by the company Chemence

### Example 4

| | |
|---|---|
| Methylheptyl cyanoacrylate (1) | 10% |
| Pure olive oil from Arista industries | 50% |
| Poly(vinyl laurate) | 39% |
| Monoethanolamine | 1% |

(1) sold by the company Chemence

### Example 5

| | |
|---|---|
| Ethoxyethyl cyanaoacrylate (1) | 60% |
| Poly(vinyl laurate) | 39% |
| Monoethanolamine | 1% |

(1) EO 460 sold by the company Tong Shen

### Example 6

| | |
|---|---|
| Butyl cyanoacrylate (1) | 60% |
| Poly(vinyl laurate) | 39% |
| Monoethanolamine | 1% |

(1) B 60 sold by the company Tong Shen

### Example 7

| | |
|---|---|
| Ethylhexyl cyanoacrylate (1) | 60% |
| Poly(vinyl laurate) | 39% |
| Monoethanolamine | 1% |

(1) O-60 sold by the company Tong Shen

### Example 8

| | |
|---|---|
| Methylheptyl cyanoacrylate (1) | 54% |
| Ethylhexyl cyanoacrylate (2) | 6% |
| Poly(vinyl laurate) | 39% |
| Monoethanolamine | 1% |

(1) sold by the company Chemence
(2) O-60 sold by the company Tong Shen

### Example 9

| | |
|---|---|
| Methylheptyl cyanoacrylate (1) | 42% |
| Butyl cyanoacrylate (2) | 18% |
| Poly(vinyl laurate) | 39% |
| Monoethanolamine | 1% |

(1) sold by the company Chemence
(2) B-60 sold by the company Tong Shen

1. Use, for the cosmetic treatment of keratin materials, of a film-forming composition comprising, in a cosmetically acceptable medium, at least one electro-philic monomer and at least one non-silicone polymer such that the film obtained from the composition by drying at ambient temperature and with a relative humidity of approximately 50% has a Young's modulus of between 1 and 100 MPa, measured for a thickness of 0.5 mm and with a tensile speed of 20 mm/min.

2-39. (canceled)
